(19) United States
(12) Patent Application Publication
(10) Pub. No.: US 2026/0354805 A1
(43) Pub. Date: Nov. 12, 2026

(54) RELEASABLE COUPLING FOR A MEDICAL DEVICE AND RELATED METHODS

(71) Applicant: Boston Scientific Scimed, Inc., Maple Grove, MN (US)

(72) Inventors: Cassandre ROOS, Seattle, WA (US); Theodore SCOTT, North Andover, MA (US)

(73) Assignee: Boston Scientific Scimed, Inc., Maple Grove, MN (US)

(21) Appl. No.: 19/665,453

(22) Filed: May 1, 2026

Related U.S. Application Data
(60) Provisional application No. 63/801,271, filed on May 7, 2025.

(51) Int. Cl. A61B 10/04; A61B 90/00

(52) U.S. Cl. CPC A61B 10/04; A61B 90/03; A61B 2090/034

(57) ABSTRACT

A medical device may comprise a handle assembly. The handle assembly may include a main body, a movable actuator, a first coupling device, a second coupling device, and a distal component. The first coupling device may be configured to releasably couple the movable actuator to the main body. The second coupling device may include a collet and a collet nut, and the second coupling device may releasably couple to a distal end of the main body. The distal component may include a sheath and a wire extending through the shaft. The wire may include an enlarged portion at a proximal end of the wire. The distal component may be configured to be releasably coupled to the handle assembly such that the enlarged portion engages with the first coupling device and the sheath engages with the second coupling device.

### CROSS-REFERENCE TO RELATED APPLICATIONS

This application claims the benefit of priority to U.S. Provisional Application No. 63/801,271, filed on May 7, 2025, which is incorporated by reference herein in its entirety.

### TECHNICAL FIELD

The disclosure relates generally to a releasable coupling for medical devices. More specifically, aspects of the disclosure pertain to a releasable coupling for assembling a handle of a medical device and releasably coupling the handle to the remaining components of the medical device.

### BACKGROUND

Many endoscopic devices, including for example hemostasis clips and biopsy forceps, are currently single-use devices. Such devices often include a handle, a shaft, and a distal tip that includes an end effector. The distal tip and the sheath are inserted in the body to perform a procedure at a tissue site (e.g., obtain a tissue sample), while the handle remains outside of the body. Following a procedure, the entirety of the medical device, including the handle, shaft, and distal tip, may be disposed of.

### SUMMARY

Aspects of the disclosure relate to, among other things, a tissue collection device (e.g., for biopsy applications) facilitating access through body lumens and related methods. The examples described herein may have any of these features in any combination.

In an example, a medical device may comprise a handle assembly having an assembled configuration and a disassembled configuration. The handle assembly may include a main body, a movable actuator, a first coupling device, a second coupling device, and a distal component. The movable actuator may be configured to translate longitudinally relative to the main body in the assembled configuration. The first coupling device may be configured to releasably couple the movable actuator to the main body. The second coupling device may include a collet and a collet nut, and the second coupling device may releasably couple to a distal end of the main body. The distal component may include a sheath and a wire extending through the shaft. The wire may include an enlarged portion at a proximal end of the wire. The distal component may be configured to be releasably coupled to the handle assembly such that the enlarged portion engages with the first coupling device and the sheath engages with the second coupling device.

Any examples described herein may have any of these features alone or in any combination. The main body may include a first rail and a second rail, and each of the first rail and the second rail may extend longitudinally in a proximal to distal direction and define a track therebetween, and, in the assembled configuration, the first coupling device is within the track and the movable actuator slides longitudinally along the track. The main body may include a wire channel extending longitudinally through the main body, and the collet may include a channel extending longitudinally through the main body. The collet may include a channel extending longitudinally through the collet, and the collet nut may include an inner channel extending longitudinally through the collet nut. The wire channel, the channel of the collet, and the inner channel of the collet nut are aligned to form a continuous passage for the wire when the medical device is in the assembled configuration. The sheath may extend from a distal end of the handle assembly and may radially surround the wire. The enlarged portion of the wire may be configured to be positioned within the first coupling device and the sheath may be configured to be secured within the second coupling device when the collet nut is tightened to compress the collet against the shaft. The crimp retainment mechanism may include a receptacle configured to receive and secure the enlarged portion of the wire. The crimp retainment mechanism may include an indicator on an external surface. The indicator may be oriented in a first position when the crimp retainment mechanism is in a receiving configuration to allow insertion of the enlarged portion, and in a second position when the crimp retainment mechanism is in a locked configuration to secure the enlarged portion. The collet may include a plurality of arms extending distally, and the arms may be configured to flex radially inward when compressed by the collet nut to grip the sheath. The collet nut may include a threaded portion configured to engage with a complementary threaded portion on the main body. The collet nut may include an abutment with an angled internal surface. The collet may include a channel with a tapered internal diameter. The medical device may further comprise an end effector coupled to a distal end of the shaft, and the end effector may be actuated by longitudinal movement of the wire relative to the shaft. The movable actuator may be a spool configured to slide longitudinally along the main body.

In an example, a medical device may comprise a handle assembly. The handle assembly may include a main body including a first rail and a second rail. The first rail and the second rail may define a track therebetween. The handle assembly may include a movable actuator configured to translate longitudinally along the track relative to the main body. A first coupling device may be positioned within the track and may be configured to releasably couple the movable actuator to the main body. The first coupling device may be further configured to receive an enlarged portion of a wire. The handle assembly may include a second coupling device including a collet and a collet nut. The second coupling device may be configured to be releasably coupled to a distal end of the main body and the second coupling device may be configured to secure a sheath.

Any examples described herein may have any of these features alone or in any combination. The first coupling device may comprise a crimp retainment mechanism and an indicator. The crimp retainment mechanism may include a receptacle configured to receive and secure the enlarged portion of the wire. The indicator may be positioned on an external surface of the crimp retainment mechanism. The indicator may be oriented in a first position when the crimp retainment mechanism is in a receiving configuration, and the indicator may be oriented in a second position when the crimp retainment mechanism is in a locked configuration. The collet may include a plurality of arms extending distally, and the collet nut may include an abutment with an angled internal surface configured to engage the plurality of arms and compress the plurality of arms radially inward to grip the sheath when the collet nut is tightened.

The disclosure also includes a method of assembling a medical device. The method may comprise positioning a movable actuator on a main body of a handle assembly. The method may further comprise inserting a first coupling device into a slot of the movable actuator to releasably couple the movable actuator to the main body. The method may further comprise inserting a collet of a second coupling device into a distal end of the main body, positioning a collet nut of the second coupling device over the collet, and engaging a portion of the collet nut with a complementary portion on the distal end of the main body. The method may further comprise inserting a portion of a sheath and a portion of a wire through the collet nut, through the collet, and into the main body, and the portion of the wire may include an enlarged portion. The method may further comprise engaging the enlarged portion of the wire within a receptacle of the first coupling device to secure the enlarged portion of the wire and tightening the collet nut to compress a plurality of arms of the collet radially inward to grip and secure the portion of the sheath relative to the main body.

The techniques or methods described herein may include any or all of the following features or steps in any combination. The method may further comprise engaging the enlarged portion of the wire within the receptacle of the first coupling device. The method may further comprise rotating a crimp retainment mechanism from a receiving configuration to a locked configuration and an indicator on an external surface of the crimp retainment mechanism may be oriented in a first position when the crimp retainment mechanism is in the receiving configuration. The indicator on the external surface of the crimp retainment mechanism may be oriented in a second position when the crimp retainment mechanism is in the locked configuration.

It may be understood that both the foregoing general description and the following detailed description are exemplary and explanatory only and are not restrictive of the invention, as claimed. As used herein, the terms “comprises,” “comprising,” “has,” “having,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” “Distal” refers to a direction away from an operator/toward a treatment site, and “proximal” refers to a direction toward an operator. Proximal and distal directions are labeled with arrows marked “P” and “D,” respectively, throughout various figures. The term “approximately,” or like terms (e.g., “substantially”), includes values +/- 10% of a stated value. The term “or” is used disjunctively, such that “at least one of A or B” includes (A), (B), (A and A), (A and B), (B and B), etc.

### BRIEF DESCRIPTION OF THE DRAWINGS

The accompanying drawings, which are incorporated in and constitute a part of this specification, illustrate examples of this disclosure and together with the description, serve to explain the principles of the disclosure.

FIG. 1 illustrates a perspective view of an exemplary medical device, including an enlarged view of a distal portion of the medical device, according to an embodiment;

FIG. 2 illustrates an exploded view of the handle assembly of the medical device of FIG. 1;

FIG. 3 illustrates a longitudinal cross-sectional view of the handle assembly components of FIG. 1 and the proximal portions of the wire and shaft; and

FIG. 4 illustrates a perspective view of the handle assembly of the medical device of FIG. 1 in a fully assembled configuration.

### DETAILED DESCRIPTION

Reference is now made in detail to examples of this disclosure, aspects of which are illustrated in the accompanying drawings. Wherever possible, the same reference numbers will be used throughout the drawings to refer to the same or like parts.

Embodiments of this disclosure include a medical device with a proximal component that releasably couples to a distal component. The proximal component may include a handle assembly with various components that may be assembled and disassembled. The distal component may include a shaft, an actuation wire, and an end effector, which may be releasably secured to components of the handle assembly.

Before using the medical device, the handle assembly may be in a disassembled configuration. Then the handle assembly may be transitioned to an assembled configuration for securing the distal component to the handle assembly. In the assembled configuration, the proximal portions of the wire and the sheath may be introduced into and releasably coupled to the components of the handle assembly. After a procedure, the user may transition the handle assembly back to the disassembled configuration, and the proximal portion of the wire and the sheath may be decoupled from the handle assembly. In this way, the distal component, which contacts the patient during use, may be disposed of, and the handle assembly may be sterilized and reused with a second distal component in a subsequent procedure.

Such embodiments of a medical device may result in reduced waste and reduced overall cost (e.g., fewer handles needed, less packaging). The embodiments may also result in a handle assembly that can be used with various distal components (including different end effectors).

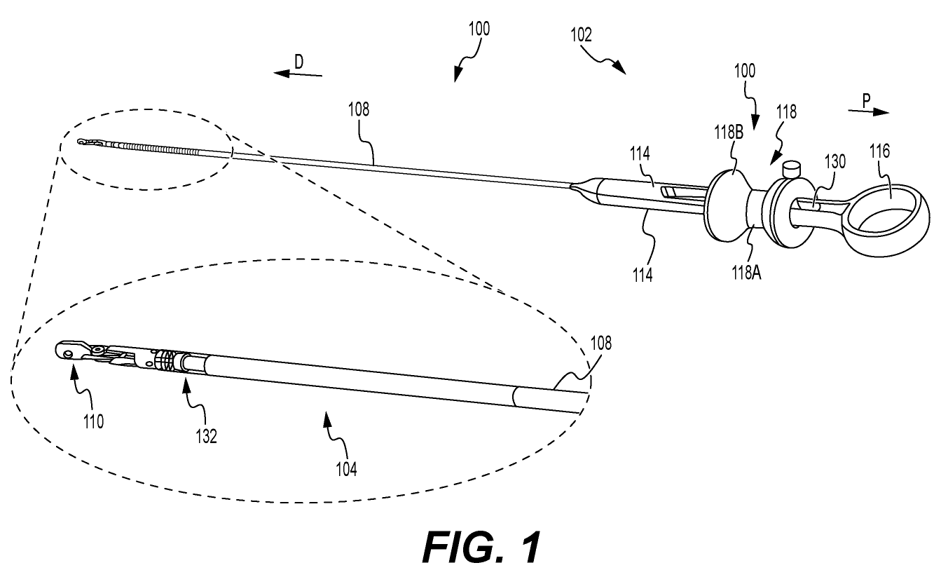

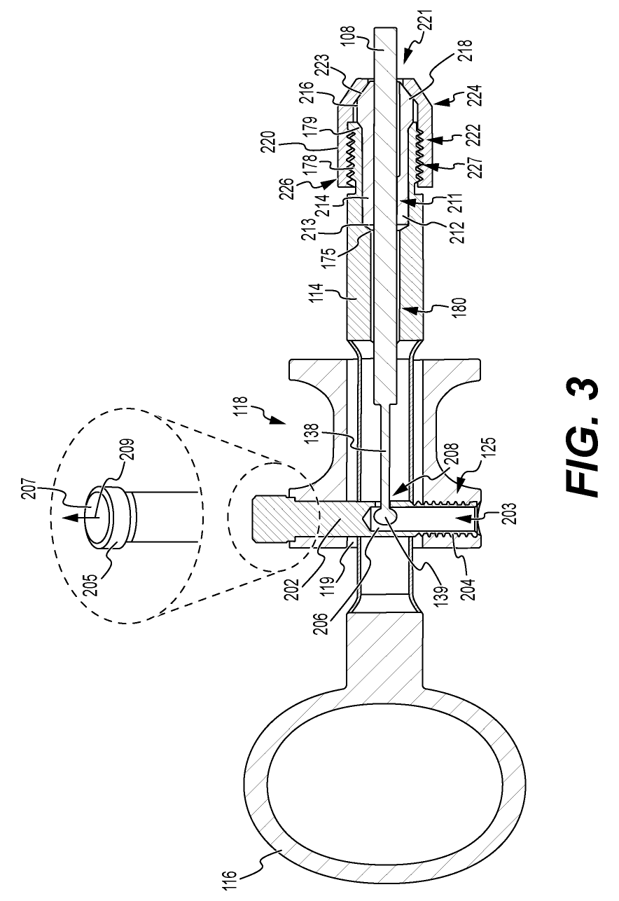

FIG. 1 illustrates a medical device 100, which includes a proximal portion 102 and a distal portion 104, with an enlarged view of the distal portion 104 shown in the figure. The medical device may include a sheath 108 and a wire 138 (see FIG. 3) connecting the proximal portion 102 to the distal portion 104. The sheath 108 is a tubular structure with sufficient length to access treatment sites within a body lumen and extends from the distal portion 104 to the proximal portion 102. The sheath 108 may include or otherwise radially surround the wire 138, which functions as a pull or actuation wire. Together, the wire 138, the sheath 108, and the distal portion 104 may constitute the distal component of the medical device 100. The wire 138 extends from the handle assembly 106 through the sheath 108. The wire 138 can be moved within and relative to the sheath 108 by manipulating aspects of the handle assembly 106, thereby controlling various aspects of the distal portion 104. As described below, an end effector 110 is positioned at the distal end of the medical device 100, specifically at the distal portion 104. In an exemplary embodiment, the distal end of wire 138 may couple to the proximal ends of two other wires. Each of those two other wires may include a distal end that connects to a corresponding tool (e.g., jaw) of the end effector 110. Translation of wire 138 will cause the two other wires to actuate the end effector 110. Alternatively, medical device 100 may include two wires 138 extending from the handle assembly 106 to the end effector 110, one wire 138 for each tool of the end effector 110.

The components of the handle assembly 106 may be constructed from stainless steel, medical-grade plastics, or other materials, for example, materials suitable to withstand tensile forces encountered during medical procedures and suitable to withstand a number of sterilizations. The handle assembly 106 may include a main body 114 with a ring 116 (e.g., a thumb ring) positioned at its proximal end. The handle assembly 106 includes a movable actuator, such as a spool 118. The spool 118 may include an indented portion 118A with a narrower lateral cross-section, and two extended portions 118B and 118C with wider lateral cross-sections. As detailed below, one of these extended portions (118B or 118C) may have a larger cross-section (greater length) to accommodate a first coupling device 200.

The indented portion 118A may be configured to accommodate one or more of the user's fingers to help control the movement of spool 118. The main body 114 may include a track 130 extending longitudinally through a section of the main body 114, beginning at a position that is distally spaced from the ring 116. The first coupling device 200 may extend into the track 130, such that the spool 118 and the coupling device 200 is movable and can slide longitudinally (both distally and proximally) along the track 130. The track 130 also defines the range of motion for the spool 118. By moving the spool 118 within the track 130 in either the proximal or distal direction, the user can actuate aspects of the end effector 110, such as opening and closing the jaws of an exemplary end effector.

The medical device 100 includes the end effector 110 at the distal portion 104. The end effector 110 may be a forceps with two opposing jaws. However, this disclosure encompasses several types of end effectors, instruments, tools, or other devices, including but not limited to: graspers, snares, baskets, hemostatic clips, staplers, ablation devices, tomes, suturing devices, needles, and knives. The end effector 110 may be coupled to the sheath 108 at the distal end, for example, at a coupling portion 132. The coupling portion 132 may incorporate different attachment mechanisms, such as a clevis, a bushing, or any other conventional structure designed to securely join the end effector 110 to the sheath 108 and the wire 138.

As will be described below, the handle assembly 106 may be assembled by a user. In the assembled configuration, the proximal portions of the wire 138 and the sheath 108 may be introduced into, and releasably coupled to, components of the handle assembly 106. Following the medical procedure, the wire 138 and the sheath 108 may be decoupled from the handle assembly 106. This design allows for disposal of the patient-contacting components (distal portion 104, sheath 108, and wire 138), while preserving the handle assembly 106 for reuse with new distal components in subsequent procedures.

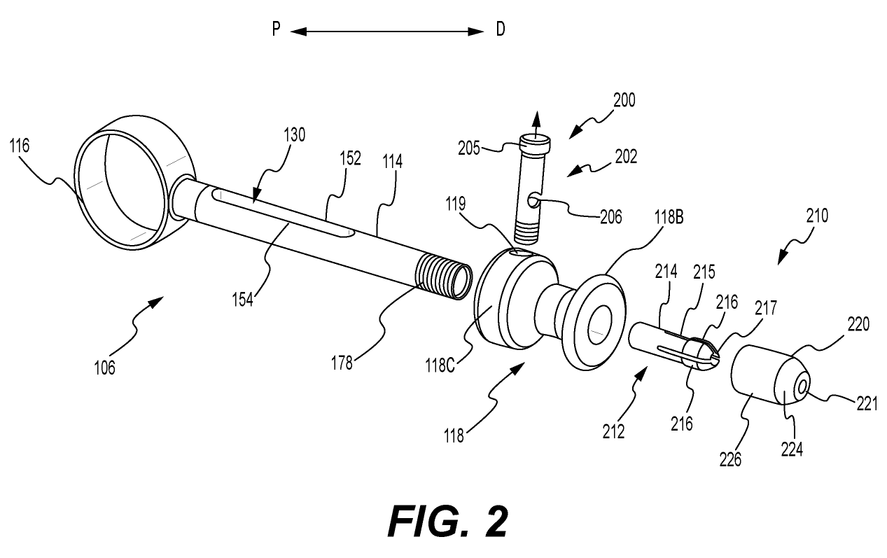

FIGS. 2 and FIG. 3 show an exploded view and a cross-sectional view of the handle assembly 106, respectively, illustrating the components that form the assembled configuration of the handle assembly 106. FIG. 2 depicts the components of the handle assembly 106 in a disassembled configuration (separated for clarity), showing the coupling devices configured to interconnect components of the handle assembly 106. The handle assembly 106 may include four primary components: the main body 114, the spool 118, a first coupling device 200, and a second coupling device 210. The following sections describe the main body 114 and the spool 118 in detail. Subsequently, the first coupling device 200, which may include a crimp retainment mechanism 202, and the second coupling device 210, which may include a collet 212 and a collet nut 220, will be described.

The main body 114 includes the ring 116 at or near the proximal end of the main body 114. The ring 116 may be integral with the main body 114 and may be any suitable shape that allows the ring 116 to be gripped by a thumb of a user, such as circular, oval, or rectangular. The main body 114 may include a first rail 152 and a second rail 154. The first rail 152 and the second rail 154 may be elongated sections of the main body 114 extending longitudinally in a proximal-to-distal direction. The first and second rails 152 and 154 may extend the length of the track 130, from a position just distal of the ring 116. In particular, the first and second rails 152, 154 may define the track 130 therebetween, with the track 130 extending longitudinally through a portion of the main body 114 from a position spaced distally from the ring 116.

The distal end of the main body 114 may include a threaded portion 178 having external threads configured to engage with corresponding threads in the proximal portion of the second coupling device 210. Throughout the disclosure, threaded connections may refer to complementary male and female threads that engage with each other, although other suitable connections (e.g., friction fit) may be used. The main body 114 defines an internal, hollow wire channel 180 positioned proximal to the threaded portion 178.

The main body 114 may include a proximal stop surface 176, proximal of the threaded portion 178, and a distal stop surface 179 at the distal end of the main body 114. Both the stop surface 176 and the distal stop surface 179 may be annular, distally-facing ledges or shoulders that extend radially inward from the inner wall of the main body 114. For example, the proximal stop surface 176 and the distal stop surface 179 may prevent proximal movement of the components of the second coupling device 210 beyond the proximal stop surface 176 and the distal stop surface 179. For example, the proximal surface 213 of the second coupling device 210 contacts the proximal stop surface 176 of the main body 114, creating a positive stop that limits further proximal movement of the second coupling device 210 relative to the main body 114. And a surface of one or more arms 216 of the second coupling device 210 contacts the distal stop surface 176 of the main body 114, creating a positive stop that limits further proximal movement of the second coupling device 210 relative to the main body 114. It is understood that an exemplary device may have one or both of the proximal stop surface 176 and the distal stop surface 179.

The wire channel 180, which may be an internal longitudinal passage extending distally from the track 130 to the distal end of the main body 114. The wire channel 180 may extend longitudinally through the center of the main body 114, beginning at the distal end of the threaded portion 178 and continuing proximally through the interior of the main body 114. The wire channel 180 is dimensioned to receive and encapsulate the wire 138 when the handle assembly 106 is assembled.

As discussed, the spool 118 mounts over the rails 152 and 154. The inner dimensions of spool 118 match the outer dimensions of rails 152 and 154 with sufficient clearance to enable relative axial movement therebetween. The spool 118 translates longitudinally along the track 130 in proximal and distal directions. This longitudinal movement of the spool 118 may control actuation of the distal end effector 110 through the wire 138 (shown in FIG. 3), which connects the spool 118 to the distal components shown in FIG. 1.

As shown throughout, the extended portion 118C of the spool 118 has a larger longitudinal dimension than the extended portion 118B to accommodate the first coupling device 200. However, in alternative embodiments, extended portion 118B could be configured with the cross-sectional size necessary to accommodate the first coupling device 200.

In the embodiment, a bore 119 is formed in the extended portion 118C and is dimensioned to allow insertion of the first coupling device 200. The bore 119 is a recessed channel that extends through the extended portion 118C, transverse to a longitudinal axis of the handle assembly 106. The depth of the bore 119 may extend entirely through the extended portion 118C, creating a through-passage that allows a crimp retainment mechanism 202 of the first coupling device 200 to be inserted from one side of the extended portion 118C and extend to the opposite side of the extended portion 118C. Furthermore, an end of the bore 119 may include a threaded portion 125 with internal threads or grooves, which may engage with a threaded portion 204 formed, for example, on an end of the crimp retainment mechanism 202.

In the embodiment, the first coupling device 200 comprises the crimp retainment mechanism 202. The crimp retainment mechanism 202 includes the threaded portion 204 dimensioned to fit within the bore 119 of the spool 118. The crimp retainment mechanism 202 may include a cylindrical head 205 extending radially outward from the extended portion 118C in an assembled configuration. The cylindrical head 205 may have a knurled or textured surface to enhance grip, helping users rotate the crimp retainment mechanism 202. The crimp retainment mechanism 202 includes a receptacle 206 that, when the handle assembly 106 is fully assembled, faces to the side of medical device 100 (for example, perpendicular to the longitudinal axis of the device 100). In the embodiment, the crimp retainment mechanism 202 may include a slot 203 configured to straddle a crimp 139. The slot 203 may be dimensioned to accommodate the crimp 139 while allowing the wire 138 to pass through to the receptacle 206. When the crimp retainment mechanism 202 is in the locked position, the slot 203 and the receptacle 206 may engage with the crimp 139, preventing proximal to distal translation of the crimp 139 relative to the crimp retainment mechanism 202.

Continuing with the description of the first coupling device 200, the side opening 208 leads to the receptacle 206 configured to removably couple a crimp 139 located at the proximal end of wire 138 to the spool 118. The crimp 139 may be an enlarged portion of the wire 138 that may be formed by compressing or deforming the proximal end of the wire 138 or otherwise adding an enlarged structure to the end of the wire 138. The crimp 139 has a diameter larger than the diameter of the wire 138, creating a mechanical stop that secures the crimp 139 within the crimp retainment mechanism 202 when engaged in the receptacle 206. The receptacle 206 and the crimp 139 may also have similar shapes (such as semi-circular or circular profiles) that prevent the wire 138 from exiting the receptacle 206 and the spool 118 during operation.

Moreover, the crimp retainment mechanism 202 may include an indicator 209 on a top face 207 of the cylindrical head 205. (See FIG. 3 showing an enlarged view of the top face 207.) In some aspects, this indicator 209 may be an arrow or other directional marking. When the crimp retainment mechanism 202 is inserted fully into the bore 119 of the spool 118, the indicator may align in a distal-axial direction relative to the wire 138. When the quarter-pin turn then is rotated/loosened one quarter turn, the indicator 209 may be oriented perpendicular to the longitudinal axis of the wire 138 and the crimp retainment mechanism 202 is in a receiving configuration (See FIG. 3.) This receiving configuration may allow insertion of the wire 138 and crimp 139 into the receptacle 206. When the crimp retainment mechanism 202 is then rotated one quarter turn back to the fully inserted position and a locked position, the indicator may align in a distal-axial direction relative to the wire 138, visually confirming secure engagement of the crimp 139 within the receptacle 206.

In an alternative embodiment, the crimp retainment mechanism 202 may include an indicator on the cylindrical head 205 that operates reversely. For example, the indicator may be oriented in a distal-axial direction relative to the wire 138 when the crimp retainment mechanism 202 is in a receiving configuration. This receiving configuration may allow insertion of the wire 138 and the crimp 139. When the crimp retainment mechanism 202 is rotated to a locked position, the indicator may align perpendicular to the longitudinal axis of the wire 138, visually confirming secure engagement of the crimp 139 within the receptacle 206. This reverse configuration may provide users with an alternative visual cue for the locked and unlocked states of the crimp retainment mechanism 202.

The second coupling device 210 may include two primary components that releasably couple to the main body 114: a collet 212 and a collet nut 220. The collet 212 includes an elongated cylindrical body 214 with multiple arms 216 extending distally. The arms 216 (e.g., four arms) may be separated by longitudinal slits 215 that extend from the distal end toward the proximal end of the collet 212, partly through a length of the collet 212, allowing the arms 216 to function as flexible cantilever-like projections. Each arm 216 may have a distal end 218 having a tapered or partial-frustoconical shape, providing a pressure-bearing surface 218’ that tapers/narrows toward the distal end. Surfaces 218’ contact/interact with the corresponding internal taper of the collet nut 220, as described below.

The collet 212 may include a channel 211 that extends longitudinally through its body. This channel 211 may be sized to accommodate both the sheath 108, the wire 138, and the crimp 139 (during assembly of the handle assembly 106). In the embodiment, the channel 211 may have a consistent diameter throughout its length. However, in some aspects, the diameter of the channel 211 may be larger at its proximal portion to allow for easier insertion of the wire 138 and the crimp 139 and may taper to a smaller diameter toward the distal end, to provide a snug fit around the sheath 108 and prevent the sheath 108 from translating relative to the collet 212, in a fully assembled configuration.

The collet 212 may include an opening 217 that may be present at the distal end of the collet 212 (See FIG. 2.) This opening 217 may serve as the entry point for the wire 138 and the crimp 139 into the channel 211 during assembly. To facilitate smooth insertion, the opening 217 may have a diameter slightly larger than a diameter of the crimp 139. When the handle assembly 106 is assembled, the channel 211 and the opening 217 may align with the wire channel 180 of the main body 114. This alignment may create a continuous passage for the wire 138 from the distal end of the medical device 100 through the collet 212 and into the main body 114.

The collet nut 220 may include a distal portion 224 and a proximal portion 226. A longitudinal inner channel 222 may run centrally through the entire collet nut 220 from its proximal end to its distal end. The proximal portion 226 may include a threaded portion 227 with internal threads configured to engage with the complementary external threads on the threaded portion 178 of the main body 114. The proximal portion 226 may have a larger diameter than the distal portion 224, which has a tapered profile that narrows in the distal direction. Tapered distal portion 224 may have an abutment 223 with an angled internal surface. When the collet nut 220 is tightened, the abutment 223 may contact the surfaces 218’ and compress the arms 216 of the collet 212, forcing the arms 216 to flex radially inward to grip the sheath 108.

The opening 221 at the distal end of the collet nut 220 has a size that permits the passage of both the wire 138 and the crimp 139, and the sheath 108. In the fully assembled configuration, the collet nut 220 and the collet 212 work in tandem to attach to the main body 114, and as a releasable coupling mechanism that firmly holds the sheath 108 in position while allowing the wire 138 to move freely through the inner channel 222 to reach the first coupling device 200 and the spool 118. When assembled, wire 138 may run through the aligned channels of multiple components: the inner channel 222 of collet nut 220, the channel 211 of the collet 212, the wire channel 180 of the main body 114, and into the spool 118. As discussed below, the wire crimp 139 at the proximal end of wire 138 may be slotted within the crimp receptacle 206 of the crimp retainment mechanism 202.

As discussed, the cylindrical head 205 of the first coupling device 200 and the collet nut 220 of the second coupling device 210 may have knurled outer surfaces to facilitate gripping and manipulation. However, various methods may be employed to tighten or adjust these coupling devices. In the embodiment, the coupling devices may be designed for toolless operation, allowing users to tighten or loosen them using only their fingers. In other aspects, the coupling devices may be configured to be tightened with specific tools. For example, the collet nut 220 may include one or more holes positioned radially around its circumference. These holes may be designed to receive a c-pin wrench, allowing for tightening and loosening of the collet nut 220. Similarly, the cylindrical head 205 of the first coupling device 200 may be adapted for use with various tools. The cylindrical head 205 may include flat surfaces or notches for accommodating a wrench or a key.

An exemplary method for assembling the handle assembly 106 and coupling the distal components of the medical device 100 to the handle assembly 106 will be described. The method of assembly may be divided into two main phases: (1) assembling the handle components by coupling the spool 118 to the main body 114 using the first coupling device 200 and coupling the second coupling device 210 to the distal end of the main body 114; and (2) coupling the distal components (wire 138, crimp 139, and sheath 108 ) to the handle assembly 106.

To begin assembly, the user may first position the spool 118 onto the main body 114 by placing the spool 118 over the first rail 152 and the second rail 154 of the main body 114. The spool 118 should be positioned over the track 130 such that the spool 118 can fully receive the crimp retainment mechanism 202 and slide longitudinally along the track 130. Next, the user may insert the crimp retainment mechanism 202 into the bore 119 of the spool 118 and through the track 130. As noted above, the crimp retainment mechanism 202 may also engage with a compatible threaded portion within the bottom of the bore 119. The crimp retainment mechanism 202 should be fully inserted with the indicator 209 on the top face 207 of the cylindrical head 205 facing distally, parallel to the longitudinal axis of the main body 114.

The user may then insert the collet 212 into the distal end of the main body 114, advancing the collet 212 proximally until the surface 213 of the body 214 of the collet 212 abuts the distal face of the stop surface 176 within the main body 114. This contact prevents the collet 212 from moving further proximally. Subsequently, the collet nut 220 may be loosely positioned onto the collet 212. The arms 216 of the collet 212 fit within the inner channel 222 of the collet nut 220. Here, the distal stop surface 179 at the distal end of the main body 114 may abut a portion of the arms 216 of the collet 212. The distal end of the collet 212 may or may not yet contact the abutment 223 of the collet nut 220. The user may partially tighten the collet nut 220 so that the abutment 223 applies light pressure or no pressure on the arms 216, so as to not compress arms 216 at this point in assembly. In addition, at this point in assembly, the external threads of the threaded portion 178 of the main body 114 may engage with the internal threads of the threaded portion 227 of the proximal portion 226 of the collet nut 220. At this stage, the collet nut 220/collet 212 (i.e., the second coupling device 210) and the crimp retainment mechanism 202 and spool 118 may remain relatively loose to allow for introduction of the distal components.

With the handle assembly components loosely assembled, the user may then rotate the crimp retainment mechanism 202 so that the indicator 209 on the cylindrical head 205 faces to the side, perpendicular to the longitudinal axis of the main body 114. As noted above, the crimp retainment mechanism 202 may include an indicator 209 (such as an arrow) on the cylindrical head 205. When the indicator 209 is oriented perpendicular (90 degrees) to the longitudinal axis of the wire 138, the receptacle 206 may be positioned to receive the crimp 139 of the wire 138, as shown in FIG. 3.

The user may then insert the wire 138 with the crimp 139 and sheath 108 through the opening 221 of the collet nut 220 and the opening 217 of the collet 212. The wire 138 continues to advance proximally through the wire channel 180 of the main body 114. The crimp 139 passes through the side opening 208 of the crimp retainment mechanism 202 and slots within the receptacle 206. The sheath 108 advances proximally until reaching a stopping point within the channel 180 of the main body 114. With the spool 118 still positioned at or near the distal end of the track 130, the user may rotate the crimp retainment mechanism 202 by 90 degrees to the locked position, which secures the crimp 139 and allows the wire 138 to move longitudinally when the spool 118 is moved by a user. Finally, the user may fully tighten the collet nut 220, compressing the arms 216 of the collet 212 until fully tightened to fully secure the sheath 108. Other orders of the above steps may be suitable for assembling the handle assembly 106 with the wire 138 and the sheath 108.

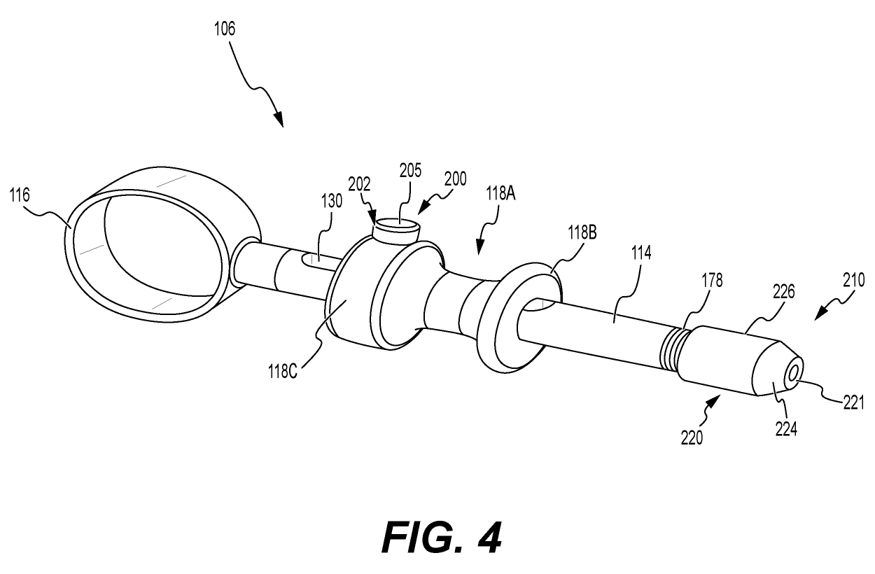

Once the user has assembled the handle assembly 106, similar to FIG. 4, the user may perform a procedure with the medical device 100. After the procedure, the user may disassemble the handle assembly 106 and remove the distal components of the medical device 100. To disassemble the medical device 100, the user may reverse the assembly steps described above, including first loosening the collet nut 220 by rotating it, which releases the compression on the arms 216 of the collet 212 and loosens the grip on the sheath 108. Next, the user rotates the crimp retainment mechanism 202 by 90 degrees to return it to the receiving configuration to allow for removal of the crimp 139. The user may then withdraw the wire 138 and the sheath 108 distally through the opening 221 of the collet nut 220. Finally, if desired, the user may completely unthread the collet nut 220 from the threaded portion 178 of the main body 114 and separate the collet 212 from the main body 114, returning the handle assembly 106 to its fully disassembled configuration for sterilization and future reuse.

While principles of this disclosure are described herein with reference to illustrative examples for particular applications, it should be understood that the disclosure is not limited thereto. Those having ordinary skill in the art and access to the teachings provided herein will recognize that additional modifications, applications, and substitutions of equivalents all fall within the scope of the examples described herein. Accordingly, the invention should not be considered limited by the foregoing description.

We claim:

1. A medical device, comprising:
a handle assembly having an assembled configuration and a disassembled configuration, the handle assembly including:
a main body;
a movable actuator configured to translate longitudinally relative to the main body in the assembled configuration;
a first coupling device configured to releasably couple the movable actuator to the main body; and
a second coupling device including a collet and a collet nut, wherein the second coupling device is configured to releasably couple to a distal end of the main body; and
a distal component including a sheath and a wire extending through a shaft, wherein the wire includes an enlarged portion at a proximal end of the wire, wherein the distal component is configured to be releasably coupled to the handle assembly such that the enlarged portion engages with the first coupling device and the sheath engages with the second coupling device.

2. The medical device of claim 1, wherein the main body includes a first rail and a second rail, wherein each of the first rail and the second rail extends longitudinally in a proximal to distal direction and defines a track therebetween, and, in the assembled configuration, the first coupling device is within the track and the movable actuator slides longitudinally along the track.

3. The medical device of claim 1, wherein the main body includes a wire channel extending longitudinally through the main body, wherein the collet includes a channel extending longitudinally through the collet, and wherein the collet nut includes an inner channel extending longitudinally through the collet nut, wherein the wire channel, the channel of the collet, and the inner channel of the collet nut are aligned to form a continuous passage for the wire when the medical device is in the assembled configuration.

4. The medical device of claim 1, wherein the sheath extends from a distal end of the handle assembly and radially surrounds the wire, wherein the enlarged portion of the wire is configured to be positioned within the first coupling device, and wherein the sheath is configured to be secured within the second coupling device when the collet nut is tightened to compress the collet against the shaft.

5. The medical device of claim 4, wherein the movable actuator includes a slot, and the first coupling device comprises a crimp retainment mechanism configured to be inserted into the slot.

6. The medical device of claim 5, wherein the crimp retainment mechanism includes a receptacle configured to receive and secure the enlarged portion of the wire.

7. The medical device of claim 5, wherein the crimp retainment mechanism includes an indicator on an external surface, wherein the indicator is oriented in a first position when the crimp retainment mechanism is in a receiving configuration to allow insertion of the enlarged portion, and in a second position when the crimp retainment mechanism is in a locked configuration to secure the enlarged portion.

8. The medical device of claim 1, wherein the collet includes a plurality of arms extending distally, wherein the arms are configured to flex radially inward when compressed by the collet nut to grip the sheath.

9. The medical device of claim 8, wherein the collet nut includes an abutment with an angled internal surface.

10. The medical device of claim 1, wherein the main body includes a stop surface, and wherein the collet includes a proximal surface configured to abut the stop surface when the second coupling device is coupled to the main body.

11. The medical device of claim 1, wherein the collet nut includes a threaded portion configured to engage with a complementary threaded portion on the main body.

12. The medical device of claim 1, wherein the collet nut includes an abutment configured to engage with the collet.

13. The medical device of claim 1, wherein the collet includes a channel with a tapered internal diameter.

14. The medical device of claim 1, further comprising an end effector coupled to a distal end of the shaft, wherein the end effector may be actuated by longitudinal movement of the wire relative to the shaft.

15. The medical device of claim 1, wherein the movable actuator is a spool configured to slide longitudinally along the main body.

16. A handle assembly, comprising:
a main body including a first rail and a second rail, wherein the first rail and the second rail extend longitudinally in a proximal direction to a distal direction, wherein the first rail and the second rail define a track therebetween;
a movable actuator configured to translate longitudinally along the track relative to the main body;
a first coupling device positioned within the track and configured to releasably couple the movable actuator to the main body, wherein the first coupling device is further configured to receive an enlarged portion of a wire; and
a second coupling device including a collet and a collet nut, wherein the second coupling device is configured to be releasably coupled to a distal end of the main body, wherein the second coupling device is configured to secure a sheath.

17. The handle assembly of claim 16, wherein the first coupling device comprises a crimp retainment mechanism and an indicator, wherein the crimp retainment mechanism includes a receptacle configured to receive and secure the enlarged portion of the wire, wherein the indicator is positioned on an external surface of the crimp retainment mechanism, wherein the indicator is oriented in a first position when the crimp retainment mechanism is in a receiving configuration, and wherein the indicator is oriented in a second position when the crimp retainment mechanism is in a locked configuration.

18. The handle assembly of claim 16, wherein the collet includes a plurality of arms extending distally, and wherein the collet nut includes an abutment with an angled internal surface configured to engage the plurality of arms and compress the plurality of arms radially inward to grip the sheath when the collet nut is tightened.

19. A method of assembling a medical device, the method comprising:
positioning a movable actuator on a main body of a handle assembly;
inserting a first coupling device into a slot of the movable actuator to releasably couple the movable actuator to the main body;
inserting a collet of a second coupling device into a distal end of the main body;
positioning a collet nut of the second coupling device over the collet and engaging a portion of the collet nut with a complementary portion on the distal end of the main body;
inserting a portion of a sheath and a portion of a wire through the collet nut, through the collet, and into the main body, wherein the portion of the wire includes an enlarged portion;
engaging the enlarged portion of the wire within a receptacle of the first coupling device to secure the enlarged portion of the wire; and
tightening the collet nut to compress a plurality of arms of the collet radially inward to grip and secure the portion of the sheath relative to the main body.

20. The method of claim 19, wherein engaging the enlarged portion of the wire within the receptacle of the first coupling device comprises rotating a crimp retainment mechanism from a receiving configuration to a locked configuration, wherein an indicator on an external surface of the crimp retainment mechanism is oriented in a first position when the crimp retainment mechanism is in the receiving configuration, and wherein the indicator on the external surface of the crimp retainment mechanism is oriented in a second position when the crimp retainment mechanism is in the locked configuration.
